(19) United States
(12) Patent Application Publication
(10) Pub. No.: US 2021/0154619 A1
(43) Pub. Date: May 27, 2021

(54) BIOLOGICAL SYSTEMS AND METHODS FOR AIR PURIFICATION

(71) Applicant: U-EARTH BIOTECH LTD, London (GB)

(72) Inventor: Elisabetta MAGGIO, Milano (IT)

(21) Appl. No.: 17/044,911

(22) PCT Filed: Feb. 2, 2019
(86) PCT No.: PCT/US19/16440

§ 371 Date: Oct. 2, 2020

Related U.S. Application Data
(60) Provisional application No. 62/653,624, filed on Apr. 6, 2018.

(51) Int. Cl. B01D 53/85; G01N 33/00; B01D 53/34; B03C 3/017

(52) U.S. Cl. CPC B01D 53/85; G01N 33/0047; B01D 2251/95; B03C 3/017; B01D 53/346

(57) ABSTRACT

The present invention relates to a device and a method associated with the device. With respect to the device, it is a biological Air Purifier, which captures air contaminants without limit of size and type by “electric molecular charge attraction” and then digests the captured contaminants by bio-oxidation by means of a microorganism compound additive.

### FIELD OF THE INVENTION

The invention relates generally to biological systems and methods for air purification. In some embodiments, the invention pertains to an air purification device, comprising a tank for accommodating a liquid; a bio support filter for accommodating a biomass additive and comprising an air passageway, the biomass additive configured to digest contaminants within air passing through the air passageway by bio-oxidation; a pipe system configured to allow transportation of the liquid within the tank to the bio support filter and provide moisture or humidity sufficient for the biomass additive to digest the air contaminants; one or more air inlets for allowing contaminated air to enter into the device; and one or more air outlets for discharging purified air. In some embodiments, the air purification device includes air quality monitoring means for assessing and detecting the quality of air purified by the device.

### BACKGROUND OF THE INVENTION

Currently there are a number of solutions for air purification. Most of these solutions are based on ventilation in an attempt to remove 99.9% of all air contaminants based on Clean Air Delivery Rate standards. However, known solutions fail to meet the needs of the air purification industry because solutions are measured using Clean Air Delivery Rate (CADR). CADR is a figure of merit that is the cubic feet per minute (CFM) of air that has had all the particles of a given size distribution removed. CADR takes in account only a comparison of the particles in the air inlet versus the air outlet during the first 10 minutes of operation and not the particles in the room for a long period of time. Recent scientific evidence shows that 92% of particles do not respond to ventilation therefore cannot be included in the CADR efficiency method which takes in account only the remaining 8% of coarse particulate matter.

Additional known solutions attempt to treat 99.9% of captured contaminants, but these solutions are similarly unable to meet the needs of the industry because the technologies are unfit to treat certain categories of contaminants. The weaknesses of each known technology are as follows:
HEPA filters: Since 92% of particles are smaller than 0.5 microns, and these particles are too small to respond to gravity and ventilation, existing systems that use HEPA filter technology are ineffective. Furthermore, HEPA membrane filters get saturated and lose sufficiency very rapidly and must be changed frequently causing a waste problem. Furthermore, these filters are very energy consuming and do not treat gas and VOC (Volatile Organic Compounds). Moreover, spores and pathogens captured by those system can proliferate on the filters inside the units exposing users to potential health danger.
IONIZERS: charge particles which attach to surfaces. Ionizers do not actually destroy pollutants but work by moving them while generating high level of ozone as a byproduct. Ozone is a harmful pollutant and a known lung irritant.
ACTIVATED CARBON FILTERS: activated carbon filters cannot remove fine particles like mold, dust, or pollen. The carbon bed fibers in these filters become saturated and chemicals are released back into the air, which is a known problem with these types of filters.
PHOTO Catalytic Oxydizers (PCO): in an effort to clean the air more efficiently PCO systems introduce chemicals to clean air. Known chemicals used in PCO systems include formaldehyde, a known human carcinogen (journal Building and Environment-Lexuan Zhong research), and also release tiny amounts of Ozone.

Most of the above technologies are used only for the residential market because they are unfit to treat efficiently a quantity of larger and more concentrated air contaminants in professional applications such as hospitals, industrial sites, waste treatment plants and outdoor settings.

Recent research has shown that 92% of airborne particles are smaller than 0.5 microns and do not respond to the forces of gravity or ventilation, thus requiring a settling time between 79 days and infinite. This research demonstrates that systems based on ventilation fail to be effective on most of the pollutants present in the air.

Thus, it is desired to provide a method and system that solves the disadvantages in the prior art and provides a biological system for air purification.

### SUMMARY OF THE INVENTION

It is an object of the invention to provide a device that efficiently lowers the level of every kind of air pollution simultaneously, which is membrane filter free, thus avoiding low efficiency due to build up clogging of the membrane pores and high energy consumption due to ventilation.

It is an object of the present invention to provide a biological air purifying system that relies on the Potential Energy Field (PEF) biophysics principle which is increasingly more efficient due to the fact that the smaller the particles are and do need powerful energy consuming vents. The biophysical phenomena relies on fact that every particle is charged on its outer layer, regardless of its size, and “grounds” the particles to the PEF in the device by “electrical molecular charge attraction.”

It is an object of the present invention to provide a device which destroys air pollutants in a natural, safe, sustainable and disposable manner.

It is an object of the present invention to provide a biological air purifier that has no need for filters ever to be changed since the internal bio-support has the same lifetime as the unit and can be washed and continuously reused not creating unwanted waste from exhaust filter disposal.

It is an object of the present invention to provide a device which does not create chemical byproducts. The trace elements found as a possible result of the bio-oxidation of complex compounds is completely safe and can be used as compost or thrown in the waste water treatment mains.

It is an object of the present invention to provide a device and associated method that efficiently removes all air pollutants, both indoor and outdoor by effect of the “electrical molecular charge attraction”, can detect its efficiency and naturally destroy air pollutants through the use of a low consumption natural media extracted from renewable sources.

The invention advantageously fills the aforementioned deficiencies by providing a biological system and method for air purification.

The present invention relates to a biological air purifier, which captures air contaminants without limit of size and type by “electric molecular charge attraction” and then digests the captured contaminants by bio-oxidation by means of a microorganism compound additive. The device advantageously includes air quality monitoring means for assessing the quality of air purified by the device and calculating related destruction rate expressed as a “Pure Air Index”. The collected data can be conveniently transferred to a cloud, and then to a dedicated software for analysis. The air quality monitoring allows broadcasting, in real time, several air quality parameters. For example, different contaminants can be separately detected with sensors such as, but not limited to, a humidity sensor, a temperature sensor, and odorous gas sensor, a carbon monoxide sensor, a carbon dioxide sensor, a NO2 sensor, a volatile organic compound sensor, etc.

In certain embodiments, a biological air purifier is provided which is made up of one or more of the following components: a water tank 301 (FIG. 1), an extractable permanent Bio Support Filter 300 topped with a water-plate 310 having circumferential holes 309 (FIG. 3) that allow distribution, by gravity, of water transferred from the water tank to the bio support filter, a submersible pump 307 with related pipes 305 and T shaped outlets 303 for distributing water within the device, or a water retention bio support material which retains water by shaking the device (portable version), and a vent 204 secured on the top of the central core pipe 308 of the extractable filter 300. The role of vent 204 is to extract air from the core pipe 308 and force the flow around the Bio Support Filter elements (FIG. 4) with the intent of providing oxygen, essential for digestion by Bio-oxidation of the captured contaminants, to the microorganism compound additive attached to the Bio Support Filter. The role of the vent 204 is also to create a turbulence around the device to better attract the larger particles which respond to ventilation.

This device can be used to treat air pollution in outdoor and indoor settings, in urban, industrial, medical, corporate, and residential applications.

In a first aspect the present invention provides an air purification device, comprising:
a tank for accommodating a liquid;
a bio support filter configured to accommodate a biomass additive and comprising an air passageway, the biomass additive configured to digest contaminants within air passing through the air passageway by bio-oxidation;
a pipe system configured to transport the liquid from the tank to the bio support filter, thereby providing moisture sufficient for the biomass additive to digest the contaminants and wash down undigestible matter resulting for bio-oxidation;
one or more air inlets for allowing contaminated air to enter into the device; and
one or more air outlets for discharging purified air.

In one or more embodiments, the bio support filter is shaped to include an air passageway in the form of one or more concentric channels, one or more vertical channels, or a corkscrew.

In one or more embodiments, the device further comprises an electronic control unit, the electronic control unit comprises a communication module for IOT remote control, and/or an air quality monitoring system.

In one or more embodiments, the air quality monitoring system includes one or more sensors for detecting an air quality parameter.

In one or more embodiments, the air quality parameter is selected from a Volatile Organic Compounds (VOC) sensor, one or more pollution sensors (such as particulate Matter sensors), a temperature sensor, a humidity sensor, a dust sensor, a gas sensor, an odor sensor, a radioactivity sensor, and a combination thereof. Additionally, any air quality sensors and other types of sensors can be added including localization, functioning parts remote detection for predictive maintenance, water quality and contamination destruction rate sensors.

In one or more embodiments, the air quality monitoring system includes an access point for transmitting data associated with the one or more air quality. In one or more embodiments, the data transmitted includes data for maintenance and destruction rate of particles, localization, and technologies for voice communication with a device such as Alexa or a voice-controlled device, and including transmitting data and parameters to a cloud-based server for further analysis via a dedicated software.

In one or more embodiments, the device further comprising a vent for coarse contaminants attraction and oxygenation of internal microorganisms compound to enhance bio-oxidation of captured contaminants.

In one or more embodiments, the pipe system includes a pump and a one or more pipes attached to the pump, wherein the pump configured to withdraw the liquid from the tank and to distribute the liquid in the bio support filter via the one or more pipes.

In one or more embodiments, the bio support filter includes a plate onto which the liquid is discharged from the pipes, wherein liquid flows into the bio support filter from the plate via holes within the plate.

In one or more embodiments, the plate includes a protective grid on top of which the biomass additive is disposed.

In one or more embodiments, the device further comprising an outlet tap for releasing the liquid from the tank.

In one or more embodiments, the device further comprising an electro valve for automatic liquid filling.

In one or more embodiments, the device further comprising a tank support base for providing ground support to the tank.

In one or more embodiments, the device further comprising a liquid level sensor for assessing the level of the liquid within the tank.

In one or more embodiments, the device further comprising a top cover for covering internal water sensitive components of the device.

In one or more embodiments, the device is configured to purify air from contaminants having a particle diameter smaller than 0.5 microns.

In one or more embodiments, the air is purified by grounding the suspended particles by “electrical charge attraction”.

In another aspect, the invention provides a method of purifying air, the method comprising
providing an air purification device with a biomass additive configured to digest contaminants within contaminated air;
providing an air quality monitoring system, the air quality monitoring system includes one or more air quality sensors being in communication with the air purification device and configured to detect one or more air quality parameters;
detecting and collecting data associated with the one or more air quality parameters; and
displaying the one or more air quality parameters on a dedicated dashboard software.

In one or more embodiments, the method further comprising determining a baseline for relative analysis of the one or more air quality parameters.

In one or more embodiments, the method further comprising transmitting the collected data to a cloud-based server using an access point affixed to the air purification device.

In one or more embodiments, the method further comprising analyzing the one or more air quality parameters using the software.

In one or more embodiments, the method further comprising providing an output of the analysis.

In one or more embodiments, the biomass additive is a nonpathogenic, non-genetically Modified micro-organisms consortium additive.

In one or more embodiments, the biomass additive is configured to transforming contaminants in water and carbon dioxide and elemental base if present.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

This disclosure will now provide a more detailed and specific description that will make reference to the accompanying drawings. The drawings and specific descriptions of the drawings, as well as any specific or alternative embodiments discussed, are intended to be read in conjunction with the entirety of this disclosure. The biological system for air purification may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

FIGS. 1A-1C show a top view (FIG. 1A), a side view (FIG. 1B), and a perspective view (FIG. 1C) of a Biotech Air Purifier of an embodiment of the present invention.

FIGS. 2A-2B show internal views of parts of the Bio Support Filter and Tank of FIG. 1.

FIGS. 3A-3D show internal details of the Bio Support Filter and Tank of FIG. 1.

FIG. 4 is top view of concentric circles Bio Support Shape of an embodiment of the present invention.

FIG. 5 is a top view of Multilayer Vertical Bio Support Shape of an embodiment of the present invention.

FIG. 6 is a top view of Corkscrew Bio Support Shape of an embodiment of the present invention.

FIG. 7 shows perspective views of Biotech Air Purifier model range of embodiments of the present invention.

FIG. 8 is a system overview of an embodiment of the present invention.

FIGS. 9A-9B are a front view (FIG. 9A) and a side view (FIG. 9B) of an Air Quality Monitoring System of an embodiment of the present invention.

FIG. 10 shows charts and system architecture of Air monitoring system details of an embodiment of the present invention.

FIG. 11 is a photograph of an internal configuration of the filter of an embodiment of the invention.

FIG. 12 is top view of concentric circles Bio Support Shape of FIG. 4 showing direction of airflow.

FIG. 13 is a chart of a pollution index.

FIG. 14 is a chart of real time air quality data.

FIGS. 15-16 are photographs of a software application that works with the system.

### DETAILED DESCRIPTION OF THE INVENTION

In the following description, numerous details are set forth for the purpose of explanation. However, one of ordinary skill in the art will realize that the invention may be practiced without the use of these specific details.

The present invention is directed to biological systems and methods for air purification.

The invention discloses a device for purifying air. The device is based on a biomass additive having microorganisms which can digest air contaminants by bio oxidation. The device includes a tank filled with water and a bio support filter for accommodating the biomass additive. The tank supplies moisture for the microorganisms in the bio support filter via a pump and related pipes and a vent supplies oxygen to the Bio Support Filter to enhance the digestion process of the captured contaminants by bio-oxidation while attracting also coarse particles. The bio support filter includes one or more air passageways within which contaminated air may pass and come into contact with the microorganisms.

The disclosed device is unique and advantageous when compared with other known devices and solutions because it provides a unique plug-and-play solution for every kind of contaminant regardless of particle size or type. The device is configured to purify air from contaminants including, but not limited to, pathogenic bacteria and viruses, Volatile Organic Compounds, Odorous Gases, oil mist, Mold and spores, fine dust including particles smaller than 0.5 microns, heavy metals, chemical compounds, hydrocarbons, black carbon, etc.

The unit is unique because: 1) it uses water and nonpathogenic, non GMO bacteria consortium to operate; 2) all contaminants are attracted and captured by the unit by “molecular electric charge” triggered by the Potential Energy Field naturally generated by the internal Bio Support shape and characteristic; 3) once the pollution is captured by the unit, the bacteria within the unit completely destroy the captured pollutants by bio-oxidation, naturally transforming the compounds in water and carbon dioxide such as for example: 2 C6H6+15 O2 becomes 12 CO2+6 H2O; 4) there are no membranes which get saturated compromising efficiency and no filters, lamps of cartridges to be changed; and 5) the unit relies on tap water, air and a harmless blend of non-GMO, nonpathogenic bacteria to destroy large loads of air pollution without turning it into harmful byproducts.

Similarly, the disclosed method is unique when compared with other known processes and solutions in that it: 1) combines the unit with a separate air quality sensor which independently detects air pollutants without interfering with the unit operations; 2) has and integrated localization and operational sensor system for remote assistance and predictive maintenance connected to IoT and 3) has a method of calculating carbon footprint equivalence based on operational time per unit size providing a specific Pure Air Index calculation system

This equivalence has been estimated and compared depending on the size of the unit and achieved between 276 and 6000 trees pollution removal efficiency per unit. In one or more embodiments, the herein disclosed device achieves at least about 276 trees pollution removal efficiency per unit. For example, the device achieves at least about 276, 300, 400, 500, 600, 700, 800, 900, 1000, 1500, 2000, 2500, or 3000, up to 6000 trees pollution removal efficiency per unit.

The disclosed device and system are unique in that it is structurally different from other known devices or solutions. More specifically, the device is unique due to the presence of: water, forced air flow through the bio Filter by ventilation digesting air pollution using bacteria, and an IoT Air Monitoring System.

In its most complete form, an embodiment of the invention includes a device made up of the following components: a) top cover 105 (with optionally electronic-electrical control-data communication); b) vent 204; c) water plate 310 with protective grid 203; d) Bio Support filter 300; e) pump 307 and related piping 305, 303; f) Tank 301; g) tank support base (optionally forklifted) 103; h) water outlet tap 104 or electro valve 106; i) water inlet tap 106 or electro valve, which is an inlet valve accessible from the outside cover to be connected to a water pipe (water mains); j) tank thermic coat with temperature sensors; k) biomass additive dispensers on a grid 203 attached to the cover 105; k) internal web cams; l) water level sensors (mechanical-optic or ultrasound) 306; and m) temperature-humidity-pH sensors.

These components are optionally connected as follows: the head element/top cover contains all the electronic and electrical elements which could be sensitive to water, secured in a container having appropriate wiring, and can be separated from all the other components of the unit. The vent may be a separate element secured to the Bio Support core element and connected to power the top cover element. The Bio Support can be completely removed and extracted by the tank element. Pump and related piping with faucets for water flow regulation, water level sensors, and pH sensors are connected to the Bio Support and can get detached from electron power source by fast cable connectors; and the tank and tank base are connected and are optionally provided with a tap to release water.

It should further be noted that the unit can be connected to the water mains for automatic refilling. There is no need for air ducting or hoods.

The most complete form of performing the method associated with the disclosed device consists of the following steps: install the Air Quality Monitoring System 200 for a baseline period of collection data; install and turn on the biological air purifiers; detect air quality improvements 800 through related software dashboard; display results in the installed unit network on the map in the APP.

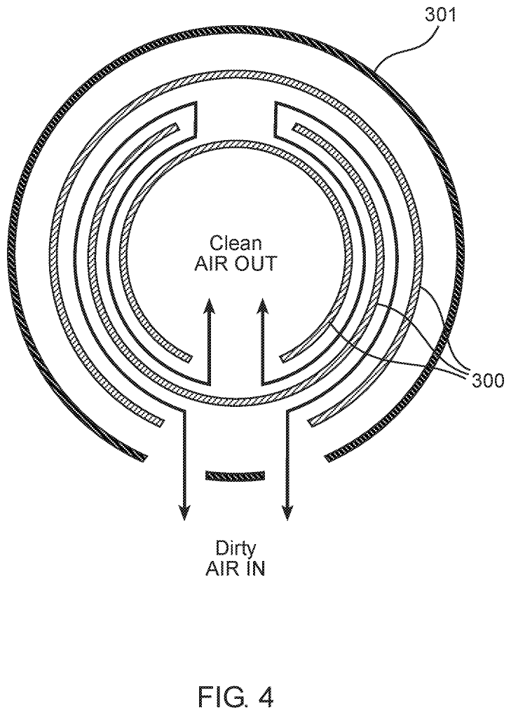

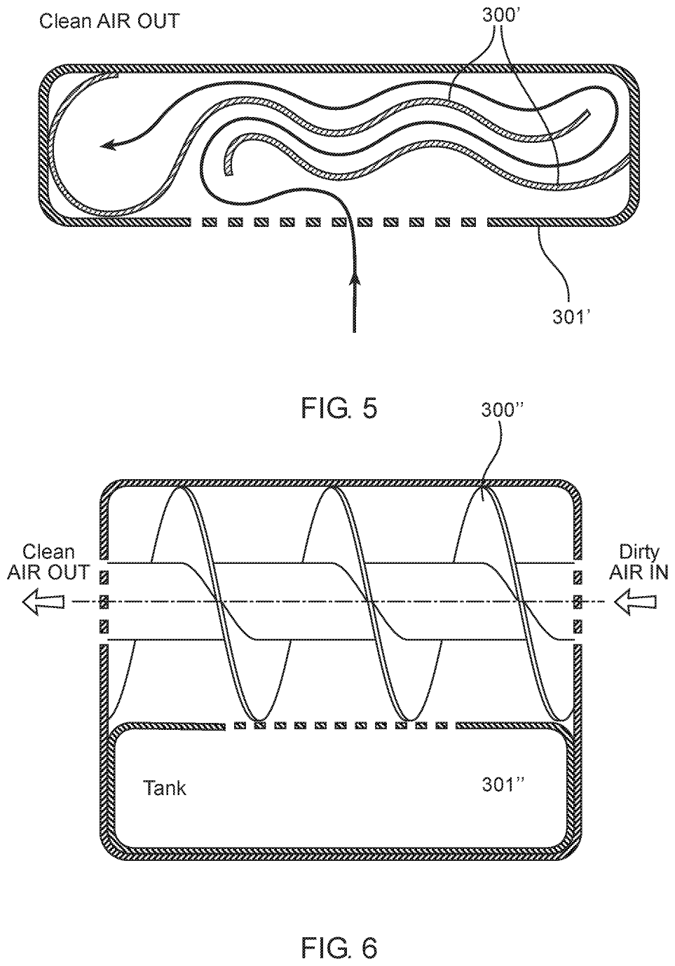

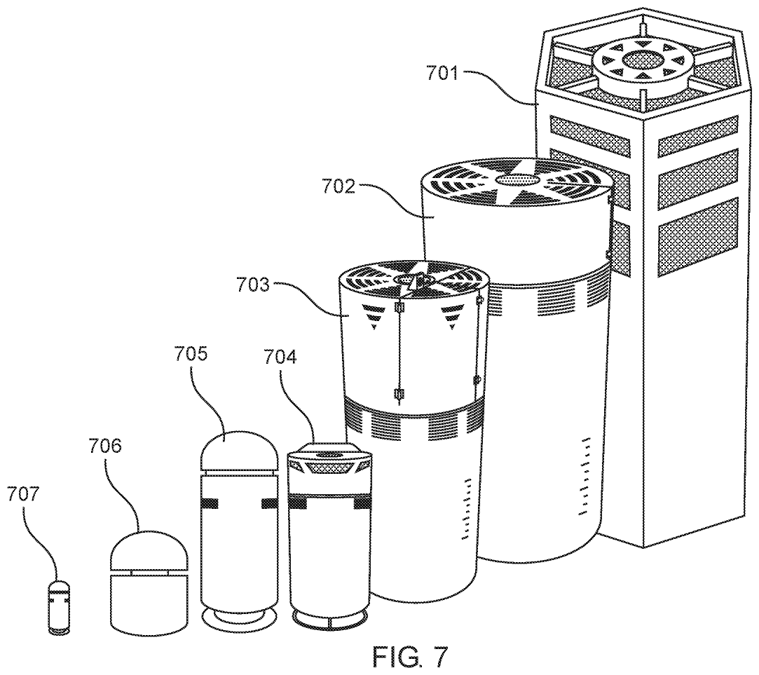

The device may also have one or more of the following variants of elements and features: the Bio Support shape can be, but is not limited to, two or more concentric circles (FIG. 4); two or more vertical multilayers (FIG. 5); and corkscrew FIG. 6.

The Bio Support shape can be made of different materials, including but not limited to: food grade plastic, activated carbon support/sponge, absorbent natural or synthetic fabric, stainless steel, or any combination of the above materials.

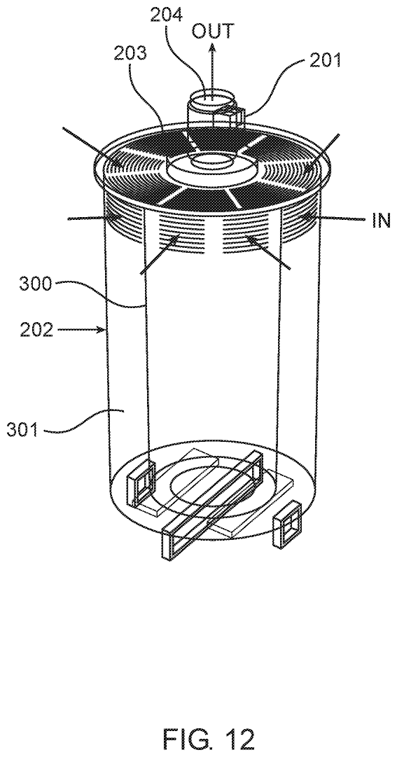

Water transmission inside/to the unit may be accomplished by the following techniques: a) water can be pulled by a submersible pump on the bottom of the tank and transferred through piping to a water plate with holes placed on top of the Bio Support in order to keep it constantly moist; and b) water can be distributed by gravity on the Bio Support by simple motion on the water plate. in the system, the water gets sucked by the pump on the bottom of the tank up through the T shaped piping to two or more T shaped outlets discharging on the water plate. The water then fills the water plate, flows through the water plate holes and along the sides of the biosupport to moist the microorganisms additive attached to the biofilter surface exposed also to air flow generated from the vent by gravity back down to the tank. (See FIG. 11).

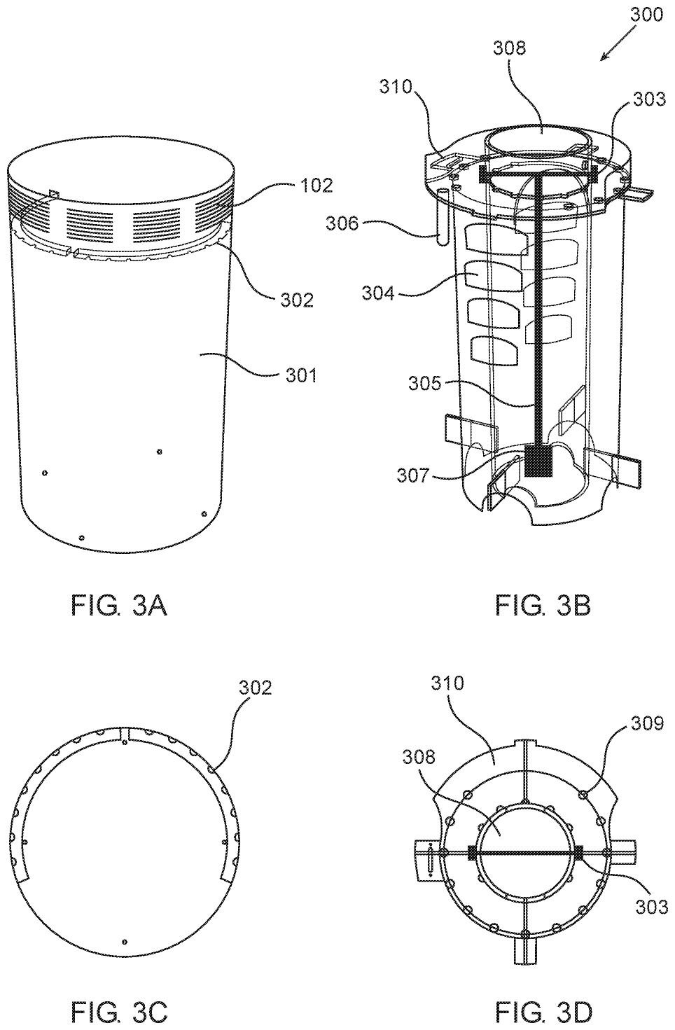

Oxygen supply to the unit internal parts can be provided by one or more vents by suction (air outlet) and forced into the bio support through related conveniently located air circulation holes (air inlets) shown in FIG. 3.

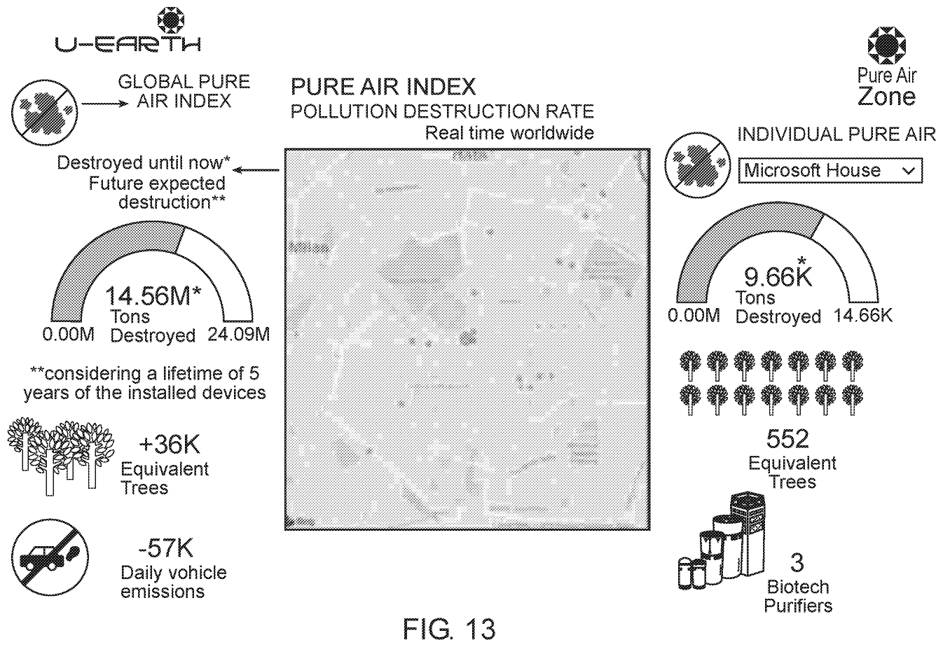

As shown in FIG. 4, the combination of air flow (tangent exposure of the bio support to oxygen) and water flow (gently washing undigestable remaining elements down to the bottom of the water tank, which happens inside/around the biofilter, is one of the key aspects to the technology. The combination of air flow and water flow creates “ideal ecosystem” (based on calculated air and water flow combined circulation, which provides a specific consortium of selected microorganisms, acting as “IMMOBILIZED cells” on the Bio Support, to greatly enhance contaminant digestion by bio-oxidation performance. FIG. 12 shows the direction of airflow into the tank.

In certain embodiments of the system the microorganisms are designed to die after 30 days and are required to be added to the water (singular-dose bottle poured in the core pipe) every 30 days. Water will evaporate by effect of bio-oxidation and (if manual) the tank must be refilled periodically (averaging every 7 days).

External materials for covering the unit may be, but are not limited to, on or more of aluminum, steel, leather, wood, fabric, plastic etc.

Operational unit features in the system may include one or more of: an electronic control of operations such as, but not limited to, a) electro valve for automated refilling 106; b) PH water sensor; c) water level control trough mechanical, optic, ultrasound or humidity sensors 306; d) remote control data transmissions to the Cloud (IoT) or Blockchain Network via GSM, LoRaWAN, WIFI, Ethernet 201.

Similarly, the associated method may also include one or more of the following steps: connection to an APP for: a) remote control of unit and Air Quality Sensors; b) pollution destruction calculation based on operational time; c) notification of unit needs such as additive addition and clean up calendar; d) location on a geographical map; e) connection in real time to after sales service team through video; and f) Augmented realty technology for units servicing aid and other useful or communication content.

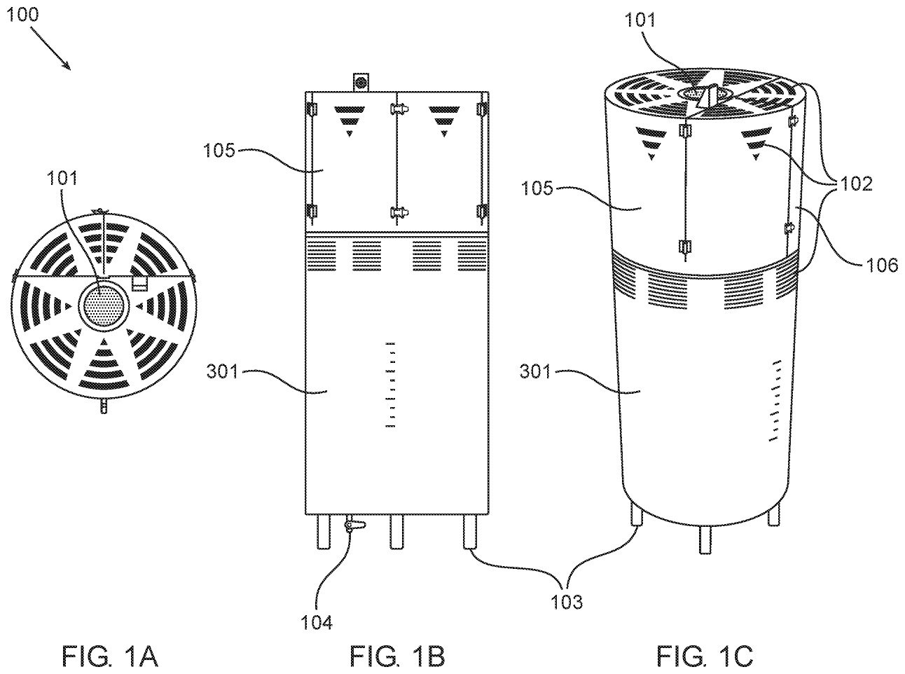

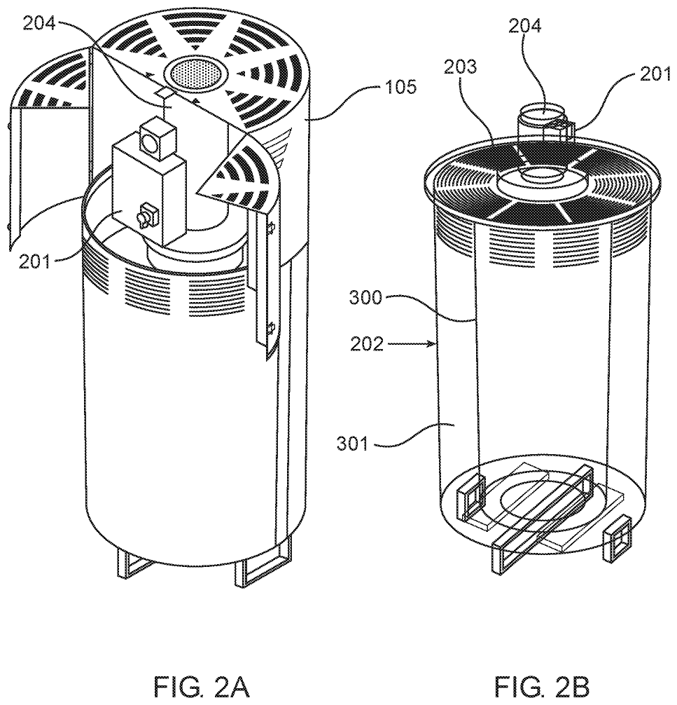

Referring now to the drawings, FIGS. 1A-1C illustrate external views of an exemplary embodiment of a device as herein disclosed. The device 100 is composed of a tank 301 placed on a base 103 and topped with a cover 105. The Air is forcefully sucked from top and lateral air inlets 102 into the internal Bio Support Filter 300 (shown in FIG. 3). Decontaminated air exits the device 100 from a top outlet 101. The tank 301 can be filled with tap water either through the water inlet equipped with an automated electronic valve 106 and connected to a water main(s), or directly poured on the internal water plate 310 (FIG. 3D) through the protective grid 203 (FIG. 2B). Water can be discharged through the tap 104 placed on the bottom of the unit. The unit 100 is designed to remain operating continuously (24/7).

Internal portions of the device 100 are demonstrated in FIGS. 2A-2D.

Opening the top cover 105, there is access to control board 201 which includes electronic control components and vent 204. The control board 201 includes a communication module to remotely control the operation of the device and have the predisposition to be integrated to existing PLC in industrial settings. The Bio Support Filter 300 is placed inside the unit tank 301. The Air Quality Monitor 200 can be connected in parallel with the unit installation to detect and track air pollution abatement performance and dynamics. A pre-dosed microorganisms additive, optionally in liquid form needs to be periodically added to the device 100, every 30 days by pouring it directly on the internal protective grid 203. The tank 301 can be filled with tap water until maximum level 202 is reached.

In FIGS. 3A-3D detailed illustration of the Bio Support Filter 300 is shown. Bio Support Filter 300 is placed inside the unit tank 301 and the vent 204 (shown in FIG. 2) is fitted and disposed on the central core pipe 308 of the filter 300. The tank 301 and Bio Support Filter 300 are two separate components of the device 100. The tank 301 has air inlet holes 102 for optimal ventilation of the Bio Support. The Bio Support Filter 300 component is equipped with a submersible pump 307 and related piping 305, a water plate 310 for water distribution on the Bio Support Filter 300, and conveniently shaped holes 304 for best fluid dynamics flow inside and throughout the Bio Support Filter 300. The water gets pumped up the piping 305 from the bottom of the tank 301 to the water plate 310 and evenly distributed by the T junction pipes 303 and back down (by gravity) wets the Bio Support 300 through the holes 309. The Bio Support Filter 300 may be additionally equipped with water level sensors 306 which can be mechanical, optic, or ultrasound. The tank 301 may be further equipped with a further drip border 302 which, once the Bio Support Filter 300 is inserted in the tank 301 the side of the tank can act a further surface for the biomass additive to stick and become active.

FIGS. 5-7 illustrate possible shapes of air pathways within the Bio Support filter 300. One possible shape of air passageway in the Bio Support filter is one or more concentric circles. The air gets sucked from the vent secured on the internal core pipe of the Bio Filter which forcefully sucks the air throughout the holes on the Bio Support layer from the external air inlet holes (FIG. 5).

Another possible shape of the Bio Support is Vertical Multilayers. The air gets sucked from the vent secured on the side core pipe of the Bio Filter which forcefully sucks the air throughout the layers of which the Bio Support is made from the external air inlet holes (FIG. 6).

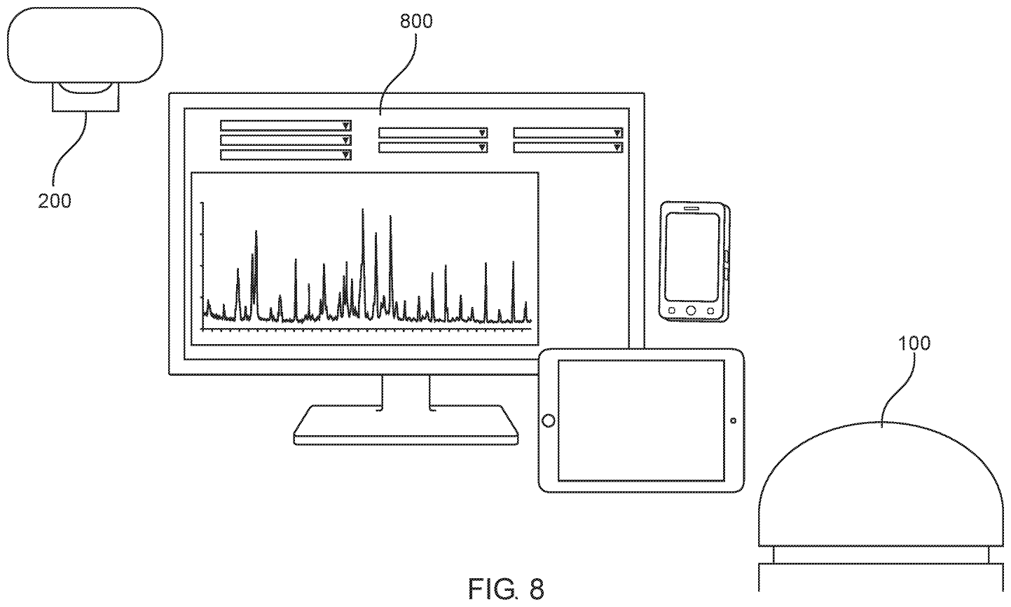

Another possible shape of the Bio Support is Corkscrew motion. The air gets sucked from the vent secured on the side core pipe of the Bio Filter which forcefully sucks the air throughout the layers of which the Bio Support is made from the external air inlet hole on the opposite side (FIG. 7).

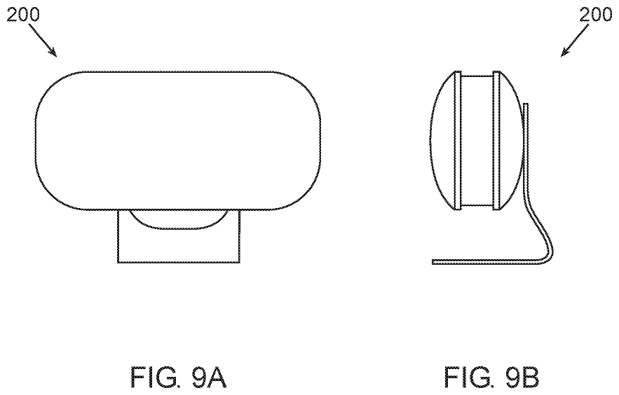

FIG. 8 illustrates air purification devices 701, 702, 703, 704, 705, 706, and 707 demonstrating various shapes, and sizes. The device can be produced in different sizes and shapes to better suit any kind of application and environment. The units may be used in urban settings (e.g., device 701), in the industry (e.g., devices 702 and 703), in a corporate setting (e.g., device 705), in the medical setting (e.g., device 704), for personal use (e.g., device 707), and in residency (e.g., device 706). The units, depending on the size and/or shape are applicable to cover a wide range of area. For example, the unit may cover an area of at least 40 square meters. The unit may cover an area in the range of at least 40 square meters and up to 70 m diameter. For example, the units are intended for use in: urban settings 701 and may include an external robust material such as stainless steel or plastic, such unit may provide protection nets for external elements such as leaves and debris and may cover up an area of 50-70 m diameter. In a further exemplary embodiment, the units may be used in industrial sites, such units may be equipped with electronic control of operations and top cover 105 protection, and may cover an area of around 500 square meters. In a further exemplary embodiment, the units may be used in the medical setting, such units may be equipped with automated water refilling system and electronic touch screen control and may cover 70-150 square meters. In yet a further exemplary embodiment, the units may be used in corporates 705, thus may be equipped with automated shut down of the unit once the water level goes below the minimum level and may cover 70-150 square meters. In yet a further exemplary embodiment, the units may be used in residential 706 suitable for countertop and may cover 40-90 square meters. In yet another exemplary embodiment the unit may be used for personal 707, and include portable unit for personal protection in car, baby stroller, train, plane, subway, office, etc. The device is used to purify immediate area surrounding the device regardless of it being in an outdoor or confined area. This is advantageous as it works in tight spaces and spaces where air is stagnant, and quality of air is required to be improved.

The units can externally be made in any possible material depending on intended field of use.

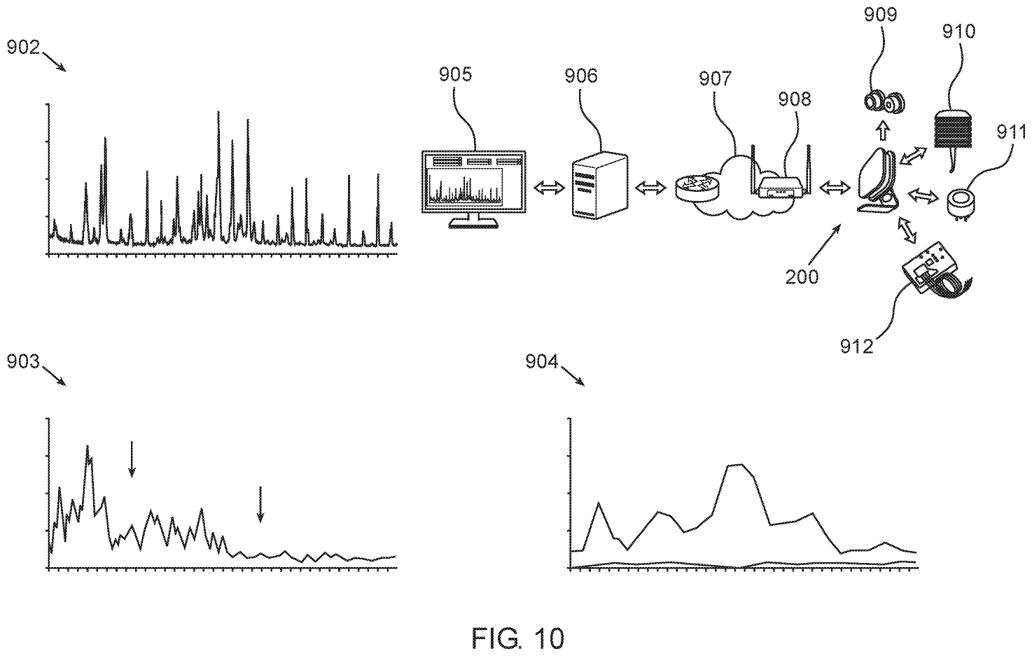

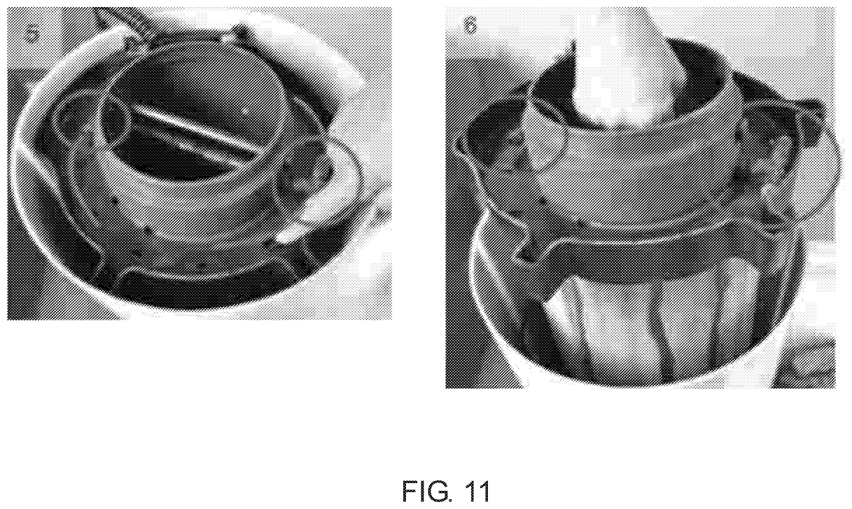

FIGS. 9-11 illustrate embodiments of the invention which include a device 100 composed of an independent Air Monitoring system 200 which collects data from specific sensors and sends them to the cloud where a software control dashboard 800 can be accessed from any mobile device or computer for interrogation confirming the performance of the device 100. The presence of the system in an area is signaled to the visitors by featuring a specific logo on the entrance door 801 and included in a WEB and APP public MAP showing location and Pure Air Index performance calculation of the installed system for marketing and sustainability purposes.

Air Quality Monitoring System 200 can detect many different parameters by including various sensors 909, 910, 911, 912, including but not limited to: VOC, NO2, CO, CO2, Odorous Gases, PM2,5, PM1, Radon, Radioactivity, Temperature, Humidity, etc. The Air Quality Monitoring System 200 can communicate the data through GSM, WIFI, Ethernet and LoRaWAN. The data sent to an access point 908 through the internet 907 and is stored on could servers 906 and consulted through a dashboard 905 where a software releases various forms of possible data aggregation 902, 903, 904.

In certain embodiments, the components of the system are installed separately. The present invention relates to a device and a method associated with the device. With respect to the device, it is a biological Air Purifier, which captures air contaminants without limit of size and type by “electric molecular charge attraction” and then digests the captured contaminants by bio-oxidation by means of a microorganism compound additive. The unit can be used in combination with a custom developed Air Quality Multiple Sensor hardware, which sends the collected data to the cloud, which is then consulted through a proprietary dashboard software. This device can be used to treat air pollution in outdoor and indoor settings, in urban, industrial, medical, corporate, and residential applications. The core components of the invention are electricity operated bioreactor where air and water circulate due to a pump and a fan, a biological non-GMO, non-pathogenic pollution eating compound to be added periodically to the bioreactor and a device which includes various Air Quality sensors connected to the Cloud. The device includes a monitoring device is set up to control in real time several Air Quality parameters then elaborated by a software in cloud for consultation through an interactive dashboard displaying periods, different contaminants separately detected with sensors such as—but not limited to—Humidity, temperature, PM0.1, PM2.5, Odorous Gases, Carbon Monoxide, Carbon Dioxide, NO2, Volatile Organic Compounds, Radon, Radioactivity etc. An algorithm based on the maximum destruction potential by bio-oxidation of every unit, multiplied by the operational time releases an impact coefficient to compare pollution destruction efficiency between users/locations. The second core component is the installation of the bioreactor, a free-standing unit which is located close to the air pollution emission source or in the middle of an area to be treated. The combination of the biological air purifiers and conveniently located monitors can detect the rate of action of the clean air zone created by every unit or cluster of units and the dynamics of air pollution travelling to the unit and being destroyed. With respect to the device it should be further noted that the internal components of the device are where the pollution eating bacteria find the ideal conditions to perform pollution destruction by bio-oxidation. In order to carry out the method the following core steps are followed: the monitoring device set up is the first step of the system, installed in order to detect a baseline of existing air pollution conditions, the second step is the installation of the bioreactor, a free standing unit which is located close to the air pollution emission source or in the middle of an area to be treated. The continued detection of improvement and general conditions of the air quality in the area treated by the system (Bioreactor+air monitor) is used to provide a “certified” air quality level to the treated space and a related Pure Air Index showing daily and cumulative since installation destruction rate. Ultimately, at the conclusion of these steps a new Air Quality Standard can be assured for safer, low concentration exposure areas. The system can be included in sustainability reports for companies and its efficiency measures against Carbon Footprint parameters.

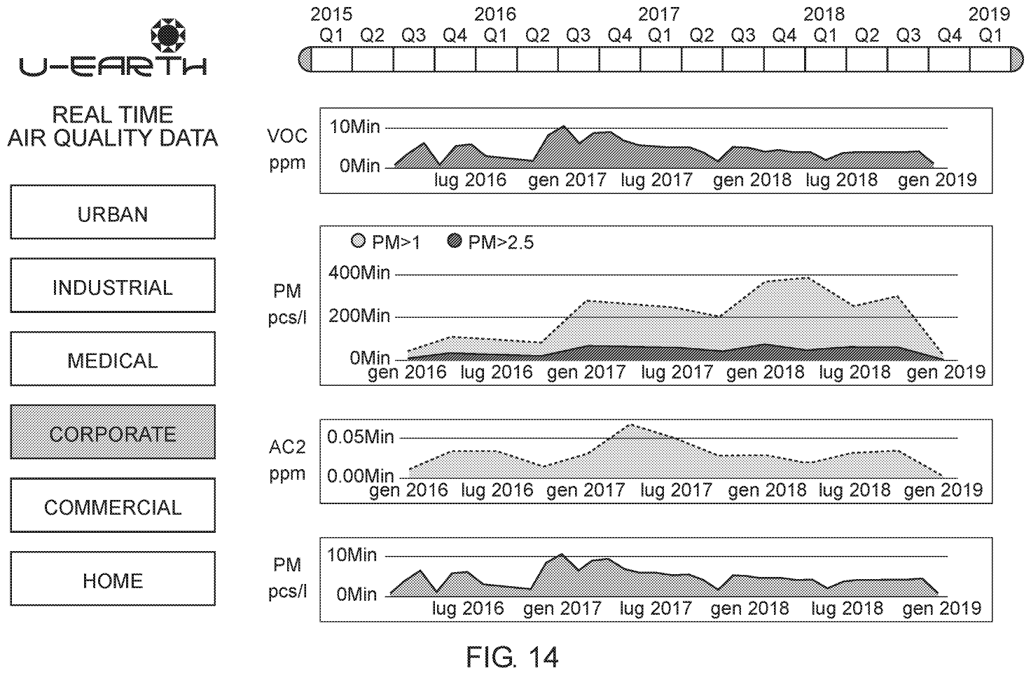

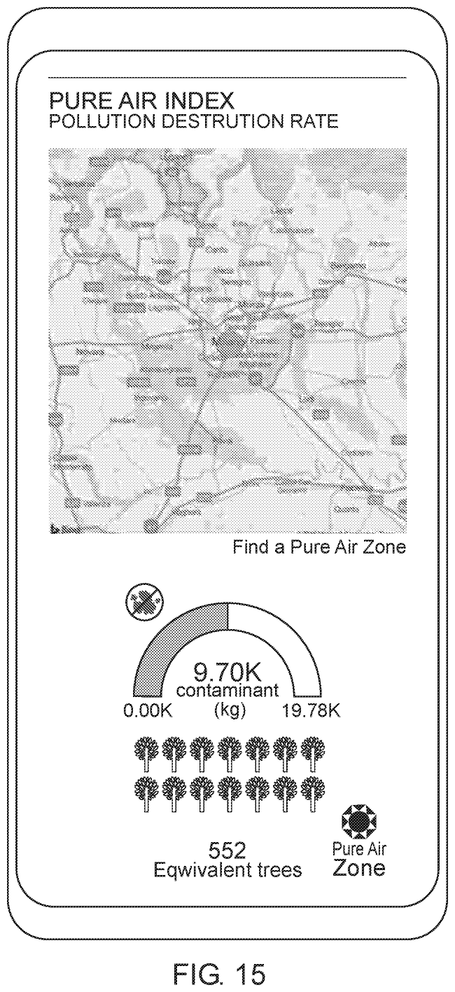

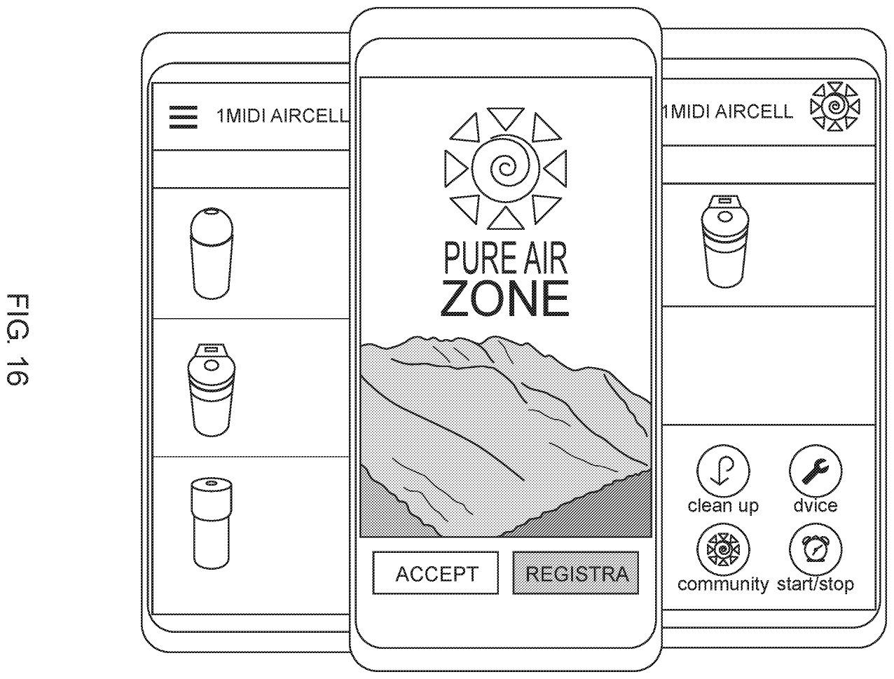

FIGS. 13-14 show a chart a of a pollution index and real time air quality data. FIGS. 15-16 are photographs of a software application that works with the system.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

1. An air purification device, comprising
a tank for accommodating a liquid;
a bio support filter configured to accommodate a biomass additive and comprising an air passageway, the biomass additive configured to digest contaminants within air passing through the air passageway by bio-oxidation;
a pipe system configured to transport the liquid from the tank to the bio support filter, thereby providing moisture sufficient for the biomass additive to digest the contaminants;
one or more air inlets for allowing contaminated air to enter into the device; and
one or more air outlets for discharging purified air.

2. The air purification device of claim 1, wherein the bio support filter is shaped to include an air passageway in the form of one or more concentric channels, one or more vertical channels, or a corkscrew.

3. The air purification device of claim 1, further comprising an electronic control unit, the electronic control unit comprises a communication module for IOT remote control, and/or an air quality monitoring system.

4. The air purification device of claim 3, wherein the air quality monitoring system includes one or more sensors for detecting an air quality parameter.

5. The air purification device of claim 4, wherein the air quality parameter is selected from a Volatile Organic Compounds (VOC) sensor, a pollution sensor, a temperature sensor, a humidity sensor, a dust sensor, a gas sensor, and a combination thereof.

6. The air purification device of claim 3, wherein the air quality monitoring system includes an access point for transmitting data associated with the air quality parameter to a cloud-based server for further analysis via a dedicated software.

7. The air purification device of claim 1, further comprising a vent to generate air flow throughout the Bio Support Filter to oxygenate microbial additive and capture larger particles in addition to the fine ones already attracted by electrical molecular charge attraction

8. The air purification device of claim 1, wherein the pipe system includes a pump and a one or more pipes attached to the pump, wherein the pump configured to withdraw the liquid from the tank and to distribute the liquid in the bio support filter via the one or more pipes.

9. The air purification device of claim 8, wherein the bio support filter includes a plate onto which the liquid is collected from the water tank, discharged from the pipes, wherein liquid flows into the bio support filter from the plate via holes within the plate and back down to the tank.

10. The air purification device of claim 9, wherein the plate includes a protective grid on top of which the biomass additive is disposed.

11. The air purification device of claim 1, further comprising an outlet tap for releasing the liquid from the tank.

12. The air purification device of claim 1, further comprising an electro valve for automatic liquid filling.

13. The air purification device of claim 1, wherein the device further comprises at least one of: (i) a tank support base for providing ground support to the tank; (ii) a liquid level sensor for assessing the level of the liquid within the tank; (iii) a top cover for covering internal water sensitive components of the device.

14. (canceled)

15. (canceled)

16. The air purification device of claim 1, which is configured to purify air from contaminants having a particle diameter smaller than 0.5 microns by grounding the suspended particles by “electrical charge attraction”.

17. A method of purifying air, the method comprising
providing an air purification device with a biomass additive configured to digest contaminants within contaminated air;
providing an air quality monitoring system, the air quality monitoring system includes one or more air quality sensors being in communication with the air purification device and configured to detect one or more air quality parameters;
detecting and collecting data associated with the one or more air quality parameters; and
displaying the one or more air quality parameters on a dedicated dashboard software.

18. The method of claim 17, further comprising determining a baseline for relative analysis of the one or more air quality parameters.

19. The method of claim 17, further comprising transmitting the collected data to a cloud-based server using an access point affixed to the air purification device.

20. The method of claim 17, further comprising analyzing the one or more air quality parameters using the software.

21. The method of claim 17, further comprising providing an output of the analysis.

22. The method of claim 17, wherein the biomass additive is a nonpathogenic, non-genetically Modified micro-organisms consortium additive.
